(19) United States
(12) Patent Application Publication
(10) Pub. No.: US 2013/0331671 A1
(43) Pub. Date: Dec. 12, 2013

(54) DISEASE DIAGNOSIS APPARATUS AND DISEASE DIAGNOSIS METHOD THEREOF, AND DISEASE DIAGNOSIS SYSTEM AND DISEASE DIAGNOSIS METHOD THEREOF

(71) Applicants: Youn-Tae KIM, Yuseong-gu (KR); Ji-Hwan LEE, Wando-gun (KR); Jae-Hyo JUNG, Buk-gu (KR)

(72) Inventors: Youn-Tae KIM, Yuseong-gu (KR); Ji-Hwan LEE, Wando-gun (KR); Jae-Hyo JUNG, Buk-gu (KR)

(73) Assignee: Industry-Academic Cooperation Foundation, Chosun University, Gwangju (KR)

(21) Appl. No.: 13/523,345

(22) Filed: Jun. 14, 2012

(30) Foreign Application Priority Data
Jun. 11, 2012 (KR) 10-2012-0062154

(51) Int. Cl. A61B 5/1477

(52) U.S. Cl. 600/345

(57) ABSTRACT

A disease diagnosis apparatus and a disease diagnosis method thereof, and a disease diagnosis system and a disease diagnosis method thereof are provided. The disease diagnosis apparatus includes a patch including one or more diagnosis modules collecting blood and measuring concentrations of one or more cardinal markers included in the collected blood, and a processor wirelessly transmitting measurement results to a server.

### CROSS-REFERENCE TO RELATED APPLICATIONS

This application claims the priority of Korean Patent Application No. 10-2012-0062154 filed on Jun. 11, 2012, in the Korean Intellectual Property Office, the disclosure of which is incorporated herein by reference.

### BACKGROUND OF THE INVENTION

1. Field of the Invention

The present invention relates to a disease diagnosis apparatus and a disease diagnosis method thereof, and a disease diagnosis system and a disease diagnosis method thereof.

The present invention is derived from research conducted as part of the National Research Foundation of the Korean-Public Welfare & Security R&D Program supported by the Ministry of Education, Science and Technology [Project Management No.: 2011-0021118, Project Title: Development of Technology of Signal Measurement/Processing Module and Systematization for Body-Mounted Diagnosis System].

2. Description of the Related Art

As the point-of-care testing (POCT) market has rapidly expanded, various disease diagnosis apparatuses for diagnosing diseases in homes have been developed. In general, a disease diagnosis apparatus used for diagnosing diseases in homes is very costly and may be very inconvenient in terms of diagnosing a disease in daily life.

### SUMMARY OF THE INVENTION

In the related art, a disease diagnosis apparatus, a disease diagnosis method thereof, a disease diagnosis system, and a disease diagnosis method thereof are required.

A first aspect of the present invention provides a disease diagnosis apparatus. The disease diagnosis apparatus includes: a patch including one or more diagnosis modules collecting blood and measuring concentrations of one or more cardinal markers included in the collected blood; and a processor wirelessly transmitting measurement results to a server.

A second aspect of the present invention provides a disease diagnosis method of a disease diagnosis apparatus. The disease diagnosis method of a disease diagnosis apparatus includes: collecting blood by using one or more diagnosis modules of a patch and measuring concentrations of one or more cardinal markers included in the collected blood; and wirelessly transmitting measurement results to a server.

A third aspect of the present invention provides a disease diagnosis system. The disease diagnosis system includes: a disease diagnosis apparatus including a patch including one or more diagnosis modules collecting blood and measuring concentrations of one or more cardinal markers included in the collected blood, and a processor wirelessly transmitting measurement results to a server by way of a portable terminal; and a server transmitting diagnosis result data determined based on the measurement results to the disease diagnosis apparatus by way of the portable terminal.

A fourth aspect of the present invention provides a disease diagnosis method of a disease diagnosis system. The disease diagnosis method of a disease diagnosis system includes: collecting, by a disease diagnosis apparatus, blood by using one or more diagnosis modules of a patch, measuring concentrations of one or more cardinal markers included in the collected blood, and wirelessly transmitting measurement results to a server by way of a portable terminal; and transmitting, by the server, diagnosis result data determined based on the measurement results to the disease diagnosis apparatus by way of the portable terminal.

The foregoing technical solutions do not fully enumerate all of the features of the present invention. The foregoing and other objects, features, aspects and advantages of the present invention will become more apparent from the following detailed description of the present invention when taken in conjunction with the accompanying drawings.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a diagram illustrating a structure of a disease diagnosis system and a configuration of a disease diagnosis apparatus constituting the disease diagnosis system according to an embodiment of the present invention;

FIG. 2 is a block diagram illustrating a configuration of a patch included in a disease diagnosis apparatus according to an embodiment of the present invention;

FIG. 3 is a diagram illustrating a configuration of a diagnosis module included in a patch according to an embodiment of the present invention;

FIG. 4 is a cross-sectional view of a diagnosis module of a patch according to an embodiment of the present invention;

FIG. 5 is a flow chart illustrating a process of an operating method for diagnosing a disease in a server included in the disease diagnosis system according to an embodiment of the present invention;

FIG. 6 is a flow chart illustrating a process of an operating method for diagnosing a disease in the disease diagnosis apparatus of the disease diagnosis system according to an embodiment of the present invention; and

FIG. 7 is a flow chart illustrating a process of an operating method for diagnosing a disease in a portable terminal of the disease diagnosis system according to an embodiment of the present invention.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In describing the present invention, if a detailed explanation for a related known function or construction is considered to unnecessarily divert from the gist of the present invention, such an explanation will be omitted but would be understood by those skilled in the art. In the drawings, the shapes and dimensions of elements may be exaggerated for clarity, and the same reference numerals will be used throughout to designate the same or like components.

It will be understood that when an element is referred to as being “connected to” another element, it can be directly connected to the other element or intervening elements may also be present. In contrast, when an element is referred to as being “directly connected to” another element, no intervening elements are present. In addition, unless explicitly described to the contrary, the word “comprise” and variations such as “comprises” or “comprising,” will be understood to imply the inclusion of stated elements but not the exclusion of any other elements.

Hereinafter, a disease diagnosis apparatus, a disease diagnosis method thereof, a disease diagnosis system, and a disease diagnosis method thereof will be described. In particular, a disease diagnosis apparatus attached to a human body to collect blood, generate clinical data required for diagnosing Ischemic heart disease based on the collected blood, and wirelessly transmit the generated clinical data to a medical attendant (or a regular physician), and an operating method of the disease diagnosis apparatus will be described. Through this, a general patient without specialized knowledge can quickly and easily treat his or her disease anywhere.

In the following description, Ischemic heart disease will be taken as an example of a disease able to be diagnosed by using the disease diagnosis apparatus, but the present invention may be applicable to any disease that can be diagnosed by using blood.

Also, a portable terminal described hereinafter refers to a terminal able to wirelessly communicate with the disease diagnosis apparatus. The portable terminal may include, for example, a cellular phone, a personal communication system (PCS), a personal data assistant (PDA), an international mobile telecommunications-2000 (IMT-2000) terminal, a smart phone, a notebook computer, a tablet personal computer (tablet PC), or the like.

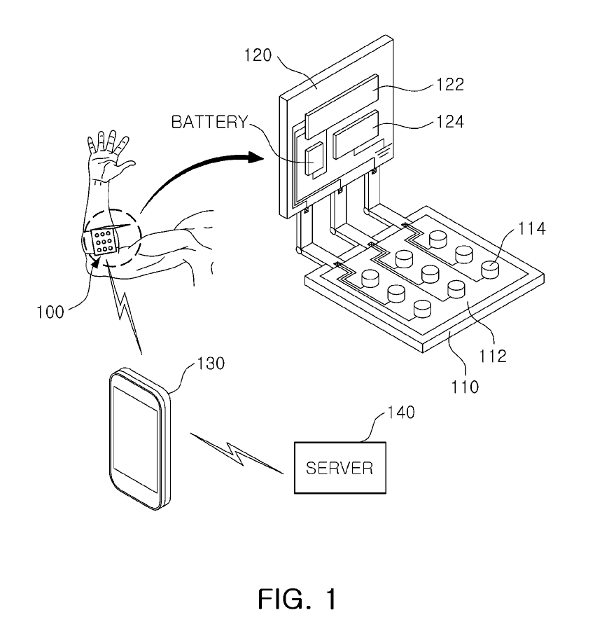

FIG. 1 is a diagram illustrating a structure of a disease diagnosis system and a configuration of a disease diagnosis apparatus constituting the disease diagnosis system according to an embodiment of the present invention.

With reference to FIG. 1, the disease diagnosis system includes a disease diagnosis apparatus 100, a portable terminal 130, and a server 140.

The disease diagnosis apparatus 100 is attached to a human body (or a user) to automatically collect blood based on pressure applied to a diagnosis module by a user's finger, analyze collected blood, and transmit analysis information to a medical attendant (or the user's regular physician) by way of the portable terminal 130 and the server 140. Thus, when the medical attendant diagnoses a disease, the disease diagnosis apparatus 100 receives information regarding the diagnosis results from the medical attendant by way of the server 140 and the portable terminal 130 and displays the same on a screen.

The portable terminal 130 transmits and receives data between the disease diagnosis apparatus 100 and the server 140.

The server 140 transmits the analysis information received from the disease diagnosis apparatus 100 by way of the portable terminal 130 to client software of the medical attendant, and information regarding the diagnosis results received from the client software to the disease diagnosis apparatus 100 by way of the portable terminal 130. Here, the medical attendant may diagnose an occurrence of Ischemic heart disease based on the information received through the client software, and provide information regarding the diagnosis results to the disease diagnosis apparatus 100 through the client software by way of the server 140 and the portable terminal 130.

Here, the disease diagnosis apparatus 100 may have a size of, for example, 110 mm×60 mm×10 mm and may be attached to a human body (e.g., the arm) by using a band, or the like. The disease diagnosis apparatus 100 may include a main body 110 including a patch 112, and a sub-body 120 including a display unit (not shown) and a plurality of (e.g., three) keys (not shown). Here, the sub-body 120 is hinge-connected to the main body 110 such that it can be opened and closed.

The patch 112 provided on the main body 110 collects blood and generates clinical data required for diagnosing Ischemic heart disease based on the collected blood, and to this end, the patch 112 includes one or more (e.g., nine (9)) diagnosis modules 114. The one or more diagnosis modules 114 collect blood and measure a concentration of one or more cardinal markers in the collected blood. Measurement results obtained therefrom are wirelessly transmitted by an internal processor 122 as clinical data required for diagnosing Ischemic heart disease to the medical attendant in real time. Here, the cardinal markers include at least one of myoglobin, creatine kinase-myocardial band (CK-MB), troponin T, and troponin I generated in blood when the symptoms of Ischemic heart disease appear.

The display unit (not shown) provided in the sub-body 120 displays information regarding the diagnosis results on a screen under the control of the internal processor 122, and the plurality of keys (not shown) provide key input data corresponding to a key pressed by the user to the internal processor 122. Accordingly, the internal processor 122 may change the format of the information displayed on the display unit (not shown) based on the key input data provided from the plurality of keys (not shown). For example, the plurality of keys (not shown) may include a first key for displaying information in the form of a graph on the display unit (not shown) and a second key for displaying information in the form of text on the display unit (not shown). The plurality of keys may further include a third key for calling the medical attendant.

The interior of the sub-body 120 is comprised of the processor 122 and a radio frequency (RF) module 124. The processor 122, which may be implemented as an advanced RISC machines (ARM) processor, processes an unprocessed signal provided from the patch 112 and wirelessly transmits the processed signal to the portable terminal 130 through the RF module 124 in real time. If the disease diagnosis apparatus 100 and the portable terminal 130 are available for short-range communication (e.g., Bluetooth™), signals may be transmitted and received between the disease diagnosis apparatus 100 and the portable terminal 130 through short-range communication. Here, the signal (i.e., clinical data) transmitted to the portable terminal 130 may be transmitted to the server 140, and accordingly, the medical attendant may diagnose Ischemic heart disease based on the clinical data from the disease diagnosis apparatus 100 and provide information regarding the diagnosis results to the disease diagnosis apparatus 100 by way of the server 140 and the portable terminal 130. When the information regarding the diagnosis results has been received, the processor 122 may output the information regarding the diagnosis results on the display unit (not shown).

Here, the processor 122 may be comprised of an analog circuit and an analog-to-digital converter (ADC) processing the unprocessed signal provided from the patch 112. The analog circuit may be comprised of an amplifying unit amplifying a pA micro-current signal provided from the patch 112 and a filter filtering the amplified signal to protect the signal against noise. The ADC converts the filtered analog signal into a digital signal. The converted digital signal may be transmitted to the server 140 by way of the portable terminal 130 through the RF module 124.

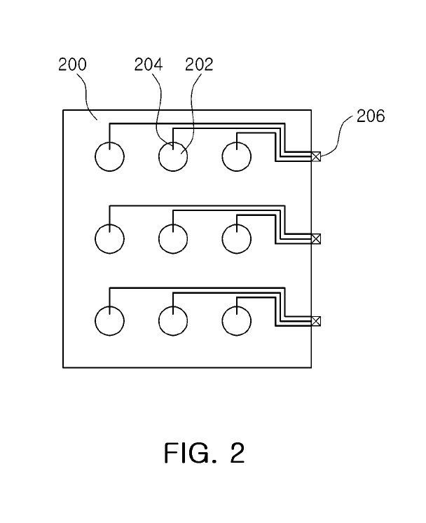

FIG. 2 is a block diagram illustrating a configuration of a patch of a disease diagnosis apparatus according to an embodiment of the present invention.

With reference to FIG. 2, a patch 200 included in a disease diagnosis apparatus is detachable and comprised of one or more diagnosis modules 202. When the patch 200 includes a plurality of diagnosis modules 202, a plurality of diagnoses may be repeatedly performed by attaching a single patch. For example, when the patch 200 includes nine diagnosis modules 202, diagnosis may be performed a maximum of nine times with the single patch.

Respective diagnosis modules 202 collect blood by using a micro-needle based on pressure applied thereto. Thereafter, respective diagnosis modules 202 detect levels of current generated through oxidation-reduction reactions between antibodies to which respective cardinal markers are attached and an antigen in the collected blood by using a 3D electrochemical sensor, and measures concentrations (i.e., current values) of the one or more cardinal markers in the collected blood, based on the detected levels of current. Thereafter, respective diagnosis modules 202 transmit the measurement results to the internal processor. Here, the one or more diagnosis modules 202 transmit the measurement results to the internal processor through individual electric wires. This is to prevent a current otherwise remaining after being generated in the already used diagnosis module from interfering with a current generated in the diagnosis module 202 when pressure is applied thereto. Here, when the oxidation-reduction reactions are completed, the diagnosis module 202 may transmit remaining blood to a waste chamber 206.

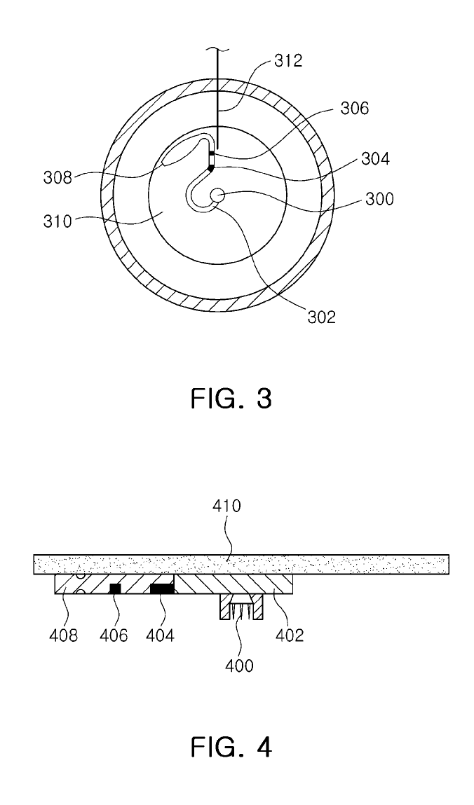

FIG. 3 is a block diagram illustrating a configuration of a diagnosis module of a patch according to an embodiment of the present invention.

With reference to FIG. 3, each of diagnosis modules included in a patch may have a size smaller than 15 mm and a sufficient pressure can be applied thereto by using the index finger of an adult man, and may be comprised of a blood reception unit and a sensor unit.

The blood reception unit may be comprised of a micro-needle 300 collecting blood based on pressure applied to the corresponding diagnosis module and a micro-fluidic chip 302 providing the collected blood to the sensor unit. Here, the micro-needle 300 is disposed in a 3×3 array and minimally penetrates skin to collect a minimal amount (e.g., 10 μl) of blood required for diagnosing a disease. Also, the micro-needle 300 made of a metal may cause skin to be infected with bacteria, so in order to prevent this, the micro-needle 300 is coated with a parylene polymer. The micro-fluidic chip 302 carries blood without the application of power thereto.

The sensor unit may be comprised of a flow-through hole (FTH) multilayer thin film 304 and a three-dimensional (3D) electrochemical sensor 306. The FTH multilayer thin film 304 may remove impurities from blood provided from the blood reception unit and provide a substrate allowing an antigen-antibody reaction to be triggered in the 3D electrochemical sensor 306. The 3D electrochemical sensor 306 detects levels of currents generated through oxidation-reduction reactions between antibodies to the respective cardinal markers are attached and the antigen in the collected blood, measure concentrations of one or more cardinal markers in the collected blood based on the detected levels of currents, and transmit the measurement results to the internal processor through the electric wire 312. Here, the 3D electrochemical sensor 306, which includes an electrode array having a 3D structure, detects an amount of current generated through an oxidation-reduction reaction between a specific antibody fixed to the electrodes and antigen in the blood by combining an antibody immobilization technique with a specific antibody technique. The antibody immobilization technique refers to a technique of fixing an antibody to an electrode, and the specific antibody technique refers to a technique of allowing an antibody to have a specific reaction to only a particular antigen. For example, in order to measure concentrations of four cardinal markers, a specific antibody corresponding to respective cardinal markers may be fixed to a predetermined position of an electrode. Also, when the oxidation-reduction reaction is completed, the 3D electrochemical sensor 306 may transmit remaining blood to the waste chamber 308.

Also, respective diagnosis modules of the patch may further include a support 310 supporting the diagnosis module so as to be maintained in parallel with the skin, such that the micro-needle 300 of the diagnosis module accurately minimally penetrates the skin.

FIG. 4 is a cross-sectional view of a diagnosis module of a patch according to an embodiment of the present invention.

With reference to FIG. 4, a micro-needle 400, a micro-fluidic chip 402, an FTH multilayer thin film 404, a 3D electrochemical sensor 406, a waster chamber 408, and a support 410 are equivalent to the micro-needle 300, the micro-fluidic chip 302, the FTH multilayer thin film 304, the 3D electrochemical sensor 306, the waster chamber 308, and the support 310, respectively, so a detailed description thereof will be omitted.

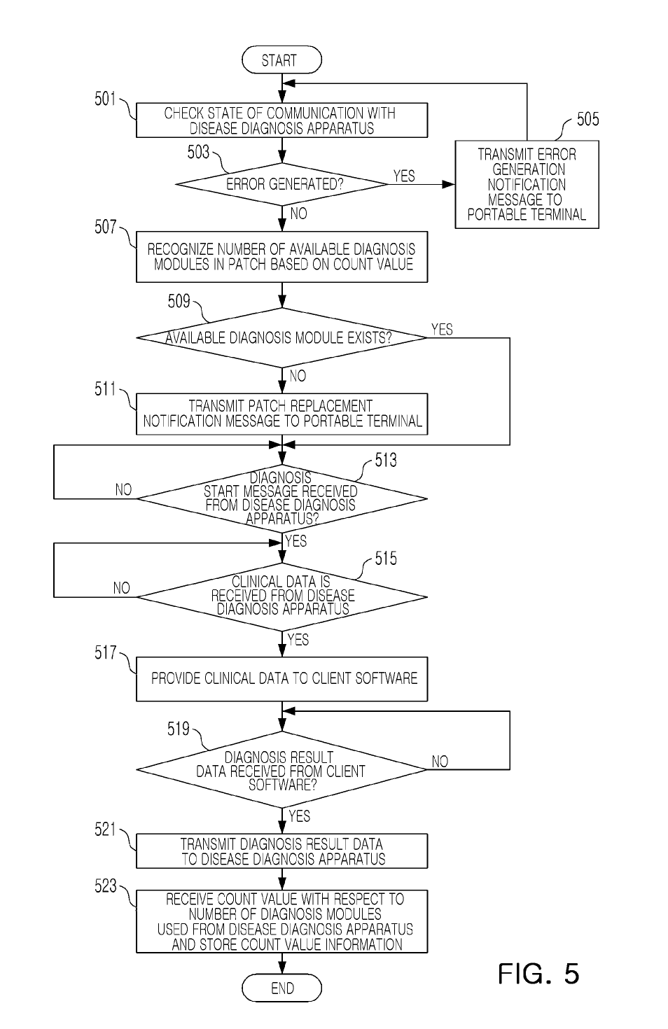

FIG. 5 is a flow chart illustrating a process of an operating method for diagnosing a disease in a server of the disease diagnosis system according to an embodiment of the present invention.

With reference to FIG. 5, the server inspects a state of communication with a disease diagnosis apparatus by way of a portable terminal in step 501. For example, the server may check a state of communication by transmitting a request message to the disease diagnosis apparatus by way of the portable terminal and determining whether or not a response message is successfully received from the disease diagnosis apparatus by way of the portable terminal, and accordingly, the server may determine whether or not there is an error in a state of communication with the disease diagnosis apparatus.

Thereafter, the server determines whether or not there is an error in a state of communication with the disease diagnosis apparatus based on the determined state of communication in step 503.

When the server detects an error in the state of communication with the disease diagnosis apparatus in step 503, the server transmits an error generation notification message to the portable terminal in step 505, and the process is returned to step 501 and the server repeatedly performs the following steps. Here, the portable terminal may display the error generation notification message on a screen thereof to inform the user that the disease diagnosis apparatus requires repairs or needs to be exchanged.

Meanwhile, if there is no error in the state of communication with the disease diagnosis apparatus in step 503, the server recognizes the number of available diagnosis modules of the patch based on a value counted by the disease diagnosis apparatus in step 507, and determines whether or not there are available diagnosis modules in the patch in step 509.

When the server determines that there is no available diagnosis module in the patch in step 509, the server transmits a patch replacement notification message to the portable terminal in step 511, and proceeds to step 513. Here, the portable terminal may display the patch replacement notification message on the screen to inform the user that the patch of the disease diagnosis apparatus is required to be changed.

Meanwhile, when there is an available diagnosis module (or modules) in the patch in step 509, the server may immediately perform step 513 to determine whether or not a diagnosis start message has been received from the disease diagnosis apparatus by way of the portable terminal.

When it is determined that a diagnosis start message has been received from the disease diagnosis apparatus in step 513, the server determines whether clinical data has been received from the disease diagnosis apparatus by way of the portable terminal in step 515.

When it is determined that clinical data has been received in step 515, the server provides the received clinical data to client software of a medical attendant in step 517.

Thereafter, the server determines whether diagnosis result data determined based on the clinical data has been received from the client software of the medical attendant in step 519.

When it is determined that diagnosis result data has been received in step 519, the server transmits the received diagnosis result data to the disease diagnosis apparatus by way of the portable terminal in step 521.

Thereafter, the server receives a count value with respect to the number of diagnosis modules used from the disease diagnosis apparatus by way of the portable terminal, and stores the received count value in step 523. The stored count value is used to recognize the number of available diagnosis modules in the patch in step 507.

Meanwhile, although not shown, the server determines whether or not GPS coordinate information of the disease diagnosis apparatus is required, based on the received diagnosis result data. When it is determined that GPS coordinate information is required, the server may transmit a GPS coordinate information request message to the disease diagnosis apparatus by way of the portable terminal. For example, when there is something wrong with a patient's condition according to the diagnosis results, the server may determine that GPS coordinate information of the disease diagnosis apparatus is required for follow-up measures such as emergency transportation to a hospital, or the like. Thereafter, when GPS coordinate information has been received from the disease diagnosis apparatus by way of the portable terminal, the server may utilize the received GPS coordinate information in taking the follow-up measures.

Also, although not shown, when it is determined that a diagnosis start message is not received in step 513, the server may determine whether or not a predetermined period of time (e.g., three hours), starting from a point in time at which a diagnosis start message was finally received, has elapsed. When the predetermined period of time (e.g., three hours), starting from a point in time at which a diagnosis start message was finally received, has elapsed, the process is returned to step 501 and the server may repeatedly perform the foregoing steps, namely, starting from step 501, and the subsequent steps.

Thereafter, the server terminates the algorithm according to an embodiment of the present invention.

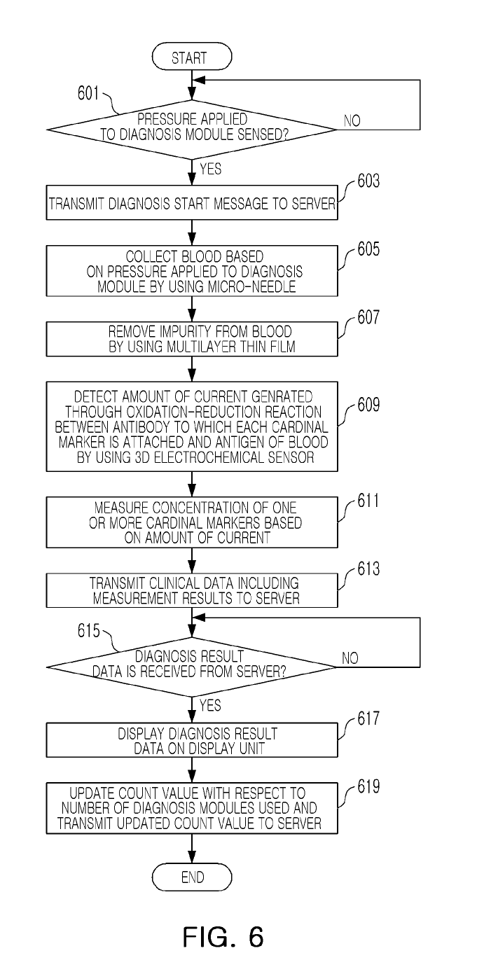

FIG. 6 is a flow chart illustrating a process of an operating method for diagnosing a disease in the disease diagnosis apparatus of the disease diagnosis system according to an embodiment of the present invention.

With reference to FIG. 6, the disease diagnosis apparatus determines whether or not pressure applied to a diagnosis module is sensed in step 601.

When pressure applied to a diagnosis module is sensed in step 601, the disease diagnosis apparatus transmits a diagnosis start message to a server by way of the portable terminal in step 603.

Next, the disease diagnosis apparatus collects blood based on the pressure applied to the diagnosis module by using a micro-needle in step 605.

Then, the disease diagnosis apparatus removes impurities from the collected blood by using a multilayer thin film in step 607.

Thereafter, the disease diagnosis apparatus detects levels of currents generated through oxidation-reduction reactions between antibodies to which respective cardinal markers are attached and the antigen in the impurity-free blood by using a 3D electrochemical sensor in step 609. For example, with respect to four cardinal markers, i.e., myoglobin, creatine kinase-myocardial band (CK-MB), troponin T, and troponin I, the levels of currents generated through oxidation-reduction reactions between antibodies to which the respective cardinal markers are attached and the antigen in the impurity-free blood may be detected.

Thereafter, the disease diagnosis apparatus measures concentrations (current values) of the one or more cardinal markers in the impurity-free blood based on the detected levels of currents in step 611.

Thereafter, the disease diagnosis apparatus transmits clinical data including the measurements results to the server by way of the portable terminal in step 613.

Thereafter, the disease diagnosis apparatus determines whether or not diagnosis result data determined based on the clinical data has been received from the server by way of the portable terminal in step 615.

When it is determined that diagnosis result data has been received in step 615, the disease diagnosis apparatus displays the received diagnosis result data on a display unit in step 617.

Thereafter, the disease diagnosis apparatus updates a count value with respect to the number of diagnosis modules used in step 619, and transmits the updated count value to the server by way of the portable terminal. For example, when a corresponding diagnosis module is used according to pressure applied to the diagnosis module, the disease diagnosis apparatus may update the count value with respect to the number of used diagnosis module by increasing 1. Here, the count value may be increased by the number of diagnosis modules of the patch.

Thereafter, the disease diagnosis apparatus terminates the algorithm according to an embodiment of the present invention.

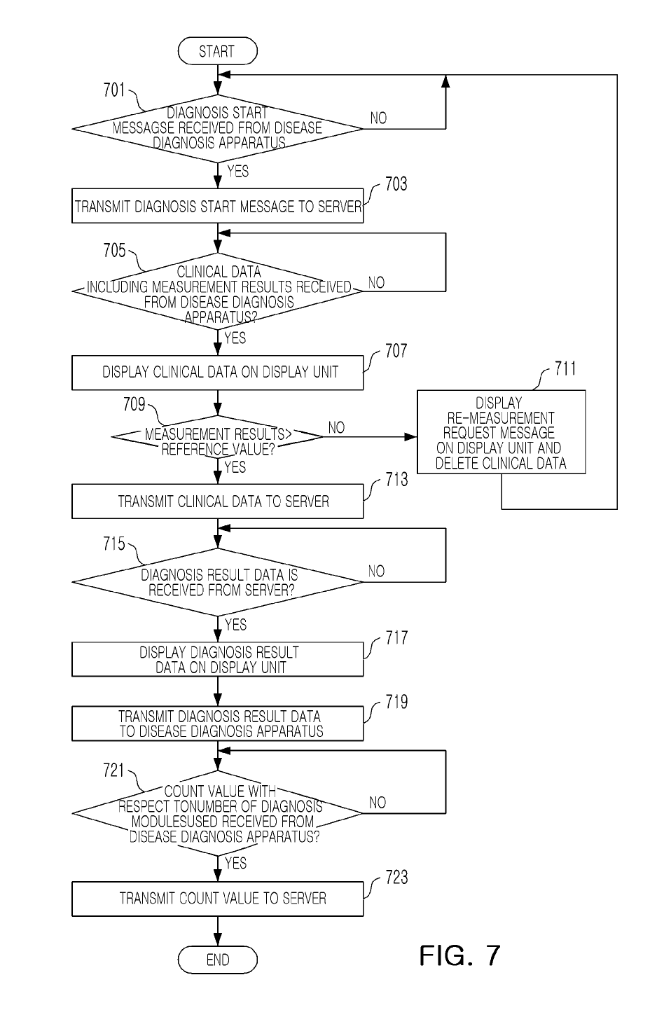

FIG. 7 is a flow chart illustrating a process of an operating method for diagnosing a disease in a portable terminal of the disease diagnosis system according to an embodiment of the present invention.

With reference to FIG. 7, the portable terminal determines whether or not a diagnosis start message has been received from the disease diagnosis apparatus in step 701.

When it is determined that a diagnosis start message has been received in step 701, the portable terminal transmits the received diagnosis start message to the server in step 703.

Thereafter, the portable terminal determines whether or not clinical data including measurement results has been received from the disease diagnosis apparatus in step 705.

When it is determined that clinical data has been received in step 705, the portable terminal displays the received clinical data on the display unit in step 707.

Thereafter, the portable terminal determines whether or not the measurement results included in the received clinical data are greater than a reference value in step 709.

When the measurement results included in the received clinical data are not greater than a reference value in step 709, the portable terminal determines that the received clinical data has a low level of reliability, and displays a re-measurement request message on the display unit and deletes the received clinical data in step 711, and returns to step 701 to repeatedly perform the foregoing steps, namely, starting from step 701, and the subsequent steps. Here, the portable terminal may inform the user that re-measurement using the disease diagnosis apparatus is required by displaying the re-measurement request message.

Meanwhile, when the measurement results included in the received clinical data are determined to be greater than the reference value in step 709, the portable terminal determines that the received clinical data has a high level of reliability, transmits the clinical data to the server in step 713, and proceeds to step 715.

The portable terminal determines whether or not diagnosis result data determined based on the clinical data has been received from the server in step 715.

When it is determined that diagnosis result data has been received in step 715, the portable terminal displays the received diagnosis result data on the display unit in step 717 and transmits the received diagnosis result data to the disease diagnosis apparatus in step 719.

Thereafter, the portable terminal determines whether or not a count value with respect to the number of diagnosis modules used has been received from the disease diagnosis apparatus in step 721.

When it is determined that a count value with respect to the number of diagnosis modules used has been received in step 721, the portable terminal transmits the received count value to the server in step 723.

Alternatively, in the place of steps 709, 711, and 713, when the measurement results included in the clinical data include concentrations (current values) measured for each of a plurality of (e.g., four) cardinal markers, the portable terminal may compare the measurement results of respective cardinal markers with a reference value, and then, when the number of cardinal markers having the measurement results greater than the reference value is greater than a majority (i.e., two), the portable terminal transmits the clinical data to the server. Meanwhile, when the number of cardinal markers having the measurement results greater than the reference value is less than the majority, the portable terminal may display a re-measurement request message on the display unit and delete the clinical data.

Thereafter, the portable terminal terminates the algorithm according to an embodiment of the present invention.

The present invention relates to a disease diagnosis apparatus allowing a general patient without specialist knowledge to easily generate clinical data required for diagnosing Ischemic heart disease at any time and in any place and transfer the same to his doctor in real time, whereby the patient can easily be diagnosed with a disease in his or her daily life, without regard to time and place and can be quickly treated. Also, through an embodiment of the present invention, Ischemic heart disease may be diagnosed in a general person or people, and can thus be prevented at an early stage, and medical expenses can be saved.

As set forth above, according to embodiments of the invention, a disease diagnosis apparatus and a disease diagnosis method thereof, and a disease diagnosis system and a disease diagnosis method thereof can be provided.

While the present invention has been shown and described in connection with the embodiments, it will be apparent to those skilled in the art that modifications and variations can be made without departing from the spirit and scope of the invention as defined by the appended claims.

What is claimed is:

1. A disease diagnosis apparatus comprising:
a patch including one or more diagnosis modules collecting blood and measuring concentrations of one or more cardinal markers included in the blood; and
a processor wirelessly transmitting measurement results to a server.

2. The disease diagnosis apparatus of claim 1, wherein the diagnosis module comprises:
a blood reception unit collecting blood based on pressure applied to the diagnosis module by using a micro-needle; and
a sensor unit sensing an amount of current generated through an oxidation-reduction reaction between an antibody to which respective cardinal markers are attached and an antigen included in the collected blood by using a three-dimensional (3D) electrochemical sensor, and measuring a concentration of each of one or more cardinal markers included in the collected blood based on the sensed amount of currents.

3. The disease diagnosis apparatus of claim 2, wherein the blood reception unit provides the collected blood to the sensor unit by using a micro-fluidic chip, and the sensor unit removes impurities included in the blood provided from the blood reception unit by using a multilayer thin film.

4. The disease diagnosis apparatus of claim 1, wherein the patch is detachable, and when the patch includes a plurality of diagnosis modules, diagnoses are repeatedly performed a plurality of times by attaching a single patch.

5. The disease diagnosis apparatus of claim 1, wherein the cardinal markers include at least one of myoglobin, creatine kinase-myocardial band (CK-MB), troponin T, and troponin I.

6. A disease diagnosis method of a disease diagnosis apparatus, the method comprising:
collecting blood by using one or more diagnosis modules of a patch and measuring concentrations of one or more cardinal markers included in the collected blood; and
wirelessly transmitting measurement results to a server.

7. The method of claim 6, wherein the collecting of blood and measuring a concentration comprises:
collecting blood based on pressure applied to the diagnosis module by using a micro-needle; and
sensing an amount of current generated through an oxidation-reduction reaction between an antibody to which respective cardinal markers are attached and an antigen included in the collected blood by using a three-dimensional (3D) electrochemical sensor, and measuring a concentration of each of one or more cardinal markers included in the collected blood based on the sensed amount of currents.

8. The method of claim 7, further comprising:
removing impurities included in the collected blood by using a multilayer thin film, before measuring the concentration thereof.

9. The method of claim 6, wherein the patch is detachable, and when the patch includes a plurality of diagnosis modules, diagnosing is repeatedly performed a plurality of times by attaching a single patch.

10. The method of claim 6, wherein the cardinal markers include at least one of myoglobin, creatine kinase-myocardial band (CK-MB), troponin T, and troponin I.

11. A disease diagnosis system comprising:
a disease diagnosis apparatus including a patch including one or more diagnosis modules collecting blood and measuring concentrations of one or more cardinal markers included in the collected blood, and a processor wirelessly transmitting measurement results to a server by way of a portable terminal; and
a server transmitting diagnosis result data determined based on the measurement results to the disease diagnosis apparatus by way of the portable terminal.

12. A disease diagnosis method of a disease diagnosis system, the method comprising:
collecting, by a disease diagnosis apparatus, blood by using one or more diagnosis modules of a patch, measuring concentrations of one or more cardinal markers included in the collected blood, and wirelessly transmitting measurement results to a server by way of a portable terminal; and
transmitting, by the server, diagnosis result data determined based on the measurement results to the disease diagnosis apparatus by way of the portable terminal.
